(19) United States
(12) Patent Application Publication
(10) Pub. No.: US 2003/0211129 A1
(43) Pub. Date: Nov. 13, 2003

(54) Self-assembled thin film coating to enhance biocompatibility of materials

(76) Inventors: William B Spillman JR., Floyd, VA (US); You-Xiong Wang, Blacksburg, VA (US); Richard O Claus, Blacksburg, VA (US)

Correspondence Address: DeWitt Ross & Stevens, Intellectual Property Department, Suite 401, 8000 Excelsior Drive, Madison, WI, 53717-1914, US

(21) Appl. No.: 10/257,814

(22) PCT Filed: Apr. 13, 2001
(86) PCT No.: PCT/US01/12042

(51) Int. Cl. A61L 2/00; B05D 1/36; A61F 2/02

(52) U.S. Cl. 424/423; 427/458; 427/402; 427/2.1; 623/926

(57) ABSTRACT

We make a substrate biocompatible by contacting it with a starting material and initiating alternating charge layer electrostatic self assembly to form a thin film. Starting materials may be poly(vinylpyrrolidone), poly{bis-(carboxylatophenoxy)phosphazene}, poly(methacrylic acid), poly(l)-lysine, poly(ethylene glycol), poly(D-glucosamine), poly(l-glutamic acid), poly(diallyldimethylamine), poly(ethylenimine), hydroxy fullerene, long-sidechain fullerene, or other polymers that participate in electrostatic self-assembly. The thin film fabrication advantageously may be at room temperature. A biocompatible thin film that is uniform and homogeneous can be provided. Optionally, ZrO₂, Al₂O₃ or TiO₂ nanoclusters also may be used in the film assembly. The film may be used in a drug delivery device or a medical device. The film may be used for tissue engineering. We also provide a biocompatible composition in which are present a plurality of layers electrostatically self-assembled from at least a polymer or fullerene asmentioned. The substrate is not particulary limited, and may be quartz, glass, plastic, metal or ceramic, a material for a bone implant, bioactive glass, polyester or other polymers, plastic or rubber tubing, bandaging material, composite material, insulator material, semi-conductor material, an artificial hip, a pacemaker, a catheter, a stent or other substrates.

### RELATED APPLICATION

This application claims priority based on U.S. application 60/197,776 filed Apr. 14, 2000.

### FIELD OF THE INVENTION

The invention generally relates to biocompatible materials, and more particularly, to making a substrate biocompatible and constructing biocompatible thin films by electrostatic self-assembly.

### BACKGROUND OF THE INVENTION

Medical and pharmaceutical technologies have developed over the years to the point that many medical conditions are treated by implanting or otherwise putting into the body a foreign object that is not naturally occurring in the body. For example, medical devices and objects made of plastic, rubber, metal, composite materials, insulator materials, semi-conductor materials or other materials are implanted to perform a particular function. Tubing used in dialysis, tubing used in heart lung machines, stents, bandaging material, artificial hips and other joints, pacemakers and catheters are examples of such internally-implanted foreign objects.

In the body, and outside of the body, such foreign objects may come in contact with body fluids, tissue, and the like, so that using artificial materials internally in the body poses many challenges. For example, the body has complex systems for recognizing “self” and “non-self” and attacking “non-self” materials found in the body. Also, other reactions occur between body materials and the implant. Implanted foreign materials in the body do not go unnoticed and most often elicit a variety of immune and other responses and reactions, interfering with the intended use of the implant and causing the patient to experience complications.

Attempting to influence the body to ignore the foreign implant, such as through drug treatments to suppress the immune and protective responses of the body to a “non-self” implant, has serious risks and disadvantages. In suppressing immunity to reduce attack on a desirable “non-self” implant, undesirable “non-self” foreign material may not receive necessary attention. Thus, a more localized approach has developed of studying and manipulating the characteristics of the foreign implant itself.

In recent years, biocompatibility technology has arisen, focusing on the acceptance of an artificial implant by the surrounding tissues and by the body as a whole. Biocompatible materials do not irritate the surrounding structure, do not provoke an abnormal inflammatory response, and do not incite allergic or immunologic reaction. Other characteristics that may be considered in a biocompatible material or device include mechanical properties (e.g., strength, stiffness and fatigue), sterilizability, manufacturability, long-term storage, and engineering design.

“Biomaterials” may be produced synthetically or biologically for use in the medical and the other fields. The use of biomaterials to interface with living systems, such as fluids, cells, and tissues of the body, has played an increasingly important role in medicine and pharmaceutics. In particular, the design of biocompatible synthetic surfaces to control the interaction between a living system and an implanted material is a major theme for biomaterial applications in medicine.

The use of devices made from biocompatible materials (biomaterials) in the treatment of patients is steadily increasing in modern healthcare. Medical devices such as pacemakers, orthopedic implants, and artificial organs are sold world-wide. Alternative drug-delivery systems that bring medication to targeted areas in the body also are widely sold.

Some substances and compounds have been identified as relatively biocompatible. Examples of biocompatible materials are certain metals, ceramics, polymers, composites and tissue-derived materials. Certain ceramics and polymers are widely used as biocompatible materials for medical devices. Relatively bioinert ceramics are typically used as structure-support implants, such as bone plates, bone screws, and femoral heads. High purity of alumina (Al₂O₃) and zirconia (ZrO₂) are among the most widely used ceramics as biocompatible materials. Titania (TiO₂) is also considered highly biocompatible. Pyrolitic carbon is considered as a biocompatible material and deposited onto finished implants. A newer form of carbon, fullerene (C₆₀) now is of interest in the scientific community.

Thus, much work has been done to try to make medical implants and devices biocompatible. A conventional approach has been to apply hydroxyapatite (HA) coatings to devices by plasma spraying and ion sputtering. However, when using a plasma spray, partial decomposition of HA undesirably occurs due to the high temperature. Another conventional approach has been to coat bioactive glass (BG) with a multilayer composition involving glass-HA/silica. Although bioactive glass is a good biocompatible material (reacting with a human physiological environment to form hydroxycarbonate apatite (HAC) on its surface), bioglass suffers from cracking in simulated body fluids, and thus may not be particularly well-suited for actual implantation.

Despite all the advances set forth above, technology for actually using biocompatible substances in the body in many cases has not yet been provided, and many biocompatible substances are undeveloped or not fully developed. Biomaterials with advantageous protein adsorption behavior remain to be developed. Medical devices that are desired to be implanted into the body remain non-biocompatible, or insufficiently biocompatible.

### SUMMARY OF THE INVENTION

It therefore is an object of this invention to provide a method of biocompatilizing a substrate, such as a substrate contained in a medical device or a drug delivery device or a substrate otherwise used internally in the body.

It is a further object of the invention to provide a method of producing certain biocompatible materials that are provided as thin films on a substrate to make the substrate more biocompatible.

Moreover, it is an object of the invention to provide biocompatible thin films that are uniform and homogeneous, and of controllable thickness.

Additionally, it is an object to employ electrostatic self-assembly (ESA) technique to incorporate various biomaterials on substrate surfaces.

A further object is to develop unique biocompatible materials with well-controlled interfaces between the living system and the implanted material.

Another object is to advance the design, synthesis, and characterization of multilayer thin films fabricated layer-by-layer by the ESA process using ceramics, polymers and fuctionalized fullerenes as candidate biomaterials.

Yet a further object is to provide thin films suitable for use in cell attachment applications and in tissue engineering.

In order to accomplish these and other objects of the invention, the present invention in a preferred embodiment provides a process of making a substrate biocompatible comprising the step of contacting a substrate having a charged surface with a starting material having an opposite charge and by electrostatic self-assembly constructing a multi-layered film of alternating charged molecular layers on the substrate, wherein the starting material is poly(vinylpyrrolidone), poly{bis(carboxylatophenoxy)phosphazene}, poly(methacrylic acid), poly(l-lysine), poly(ethylene glycol), poly(D-glucosamine), poly(l-glutamic acid), poly(diallyldimethylamine), poly(ethylenimine), hydroxy fullerene or a long-side chain fullerene (e.g., a side chain of greater than 10 carbons).

In another preferred embodiment, the present invention provides a biocompatible composition containing a plurality of layers electrostatically self-assembled from a starting material that is poly(vinylpyrrolidone), poly{bis(carboxylatophenoxy)phosphazene}, poly(methacrylic acid), poly(l-lysine), poly(ethylene glycol), poly(D-glucosamine), poly(l-glutamic acid), poly(diallyldimethylamine), poly(ethylenimine), hydroxy fullerene and long-side chain fullerene.

The present invention also in a preferred embodiment provides biocompatible materials in which a substrate and a thin film are included.

Such biocompatible compositions and biocompatible materials according to the present invention may be used in constructing medical devices and the like. In another preferred embodiment, the invention provides a drug delivery device, comprising a substrate made biocompatible by a process according to the invention and at least one drug. In a further preferred embodiment, the invention provides a biocompatible medical device made by a process according to the present invention. The present invention in another preferred embodiment provides a device for contacting a biological material, comprising a substrate; and a multilayered coating positioned on at least a portion of a surface of said substrate wherein adjacent layers of said multilayered coating are held together by ionic attraction, and wherein at least one layer of said multilayered coating is made from a material that is relatively more biocompatible than a substrate material in said substrate, whereby said multilayer coating renders the device biocompatible with said biological material.

In another preferred embodiment, the invention provides a method of rendering a device biocompatible with a biological material, comprising the step of applying a multilayered coating on at least a portion of a surface of a substrate wherein adjacent layers of said multilayered coating are held together by ionic attraction, and wherein at least one layer of said multilayered coating is made from a material that is relatively more biocompatible than a substrate material in said substrate.

### BRIEF DESCRIPTION OF THE DRAWINGS

The foregoing and other objects, aspects and advantages will be better understood from the following detailed description of the preferred embodiments of the invention with reference to the drawings, in which:

FIG. 1 shows the chemical structures of polymers used as starting materials in the present invention.

FIGS. 2 and 3 are graphs of UV-Vis spectra for thin films according to the invention.

FIGS. 4 and 5 each is an AFM image of a thin film according to the invention.

FIG. 6 is a graph of amide band intensity for different ESA thin films according to the invention.

FIGS. 7, 8 and 9 are plots of albumin adsorption onto thin film surfaces according to the invention.

FIGS. 10(a)-(d) are cross-sectional views of a thin-film being made by electrostatic self-assembly according to the present invention.

### DETAILED DESCRIPTION OF THE PREFERRED EMBODIMENTS OF THE INVENTION

In a first preferred embodiment, the invention provides a process of making a substrate biocompatible comprising the step of contacting a starting material with a substrate and initiating electrostatic self-assembly to thereby construct a thin film on the substrate.

The starting material that is subjected to an ESA process in the invention may be a polymer that is:

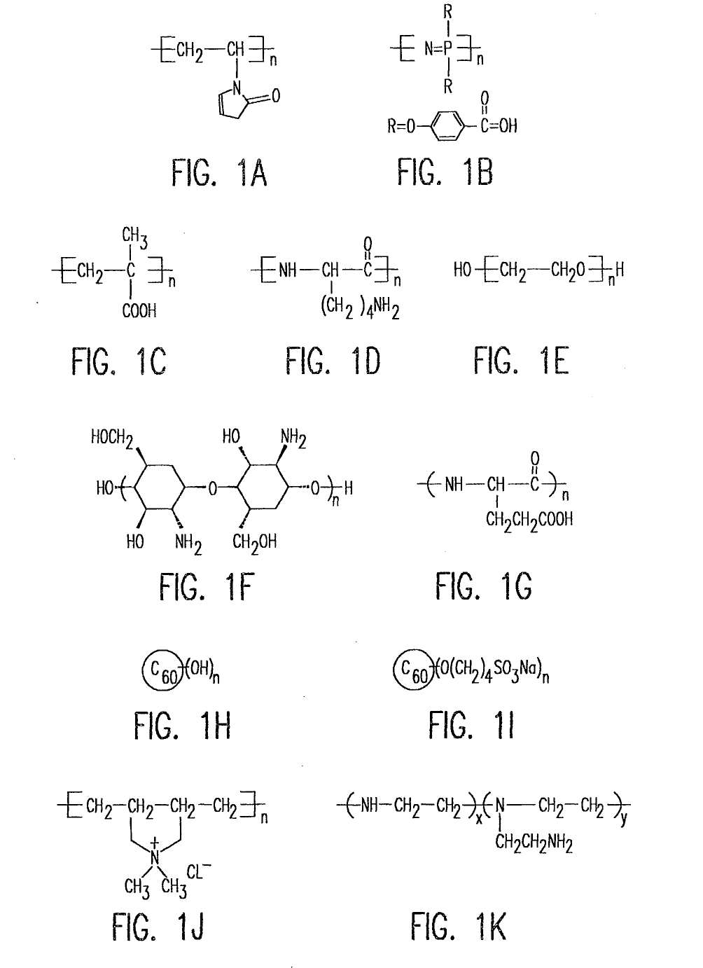

poly(vinylpyrrolidone) (“PVP”, FIG. 1(a)),

poly{bis(carboxylatophenoxy)phosphazene} (“PCPP”, FIG. 1(b)),

poly(methacrylic acid) (“PMA”, FIG. 1(c)),

poly(l-lysine) (“PL”, FIG. 1(d)),

poly(ethylene glycol) (“PEG”, FIG. 1(e)),

poly(D-glucosamine) (“chitosan”, FIG. 1(f)), or

poly(l-glutamic acid) (“PGC”, FIG. 1(g)),

poly(diallyldimethylamine) (“PDDA”, FIG. 1(j)),

poly(ethylenimine) (“PEI”, FIG. 1(k)),

or the starting material may be a fullerene that is:

hydroxy fullerene (FIG. 1(h)) or

long-sidechain fullerene (FIG. 1(i)).

Additionally, a combination of starting materials may be used.

The polymer starting materials of FIGS. 1(a)-(g) and (j)-(k) are commercially available.

For the fullerene starting materials of FIGS. 1(h)-(i), commercially available materials may be used, preferably after being subjected to minor processing.

By “hydroxy fullerene”, polyhydroxylated fullerene also is included. Polyhydroxylated fullerene 3 can be synthesized by the procedure of L. Y. Chiagn, L-Y. Wang, J. W. Swirczewski, S. Soled, S. Cameron, “Efficient synthesis of polyhydroxylated fullerene derivatives via hydrolysis of polycyclosulfated precursors,” J. Org. Chem., 59, 3960-8, 1994. The synthetic outline is as follows:

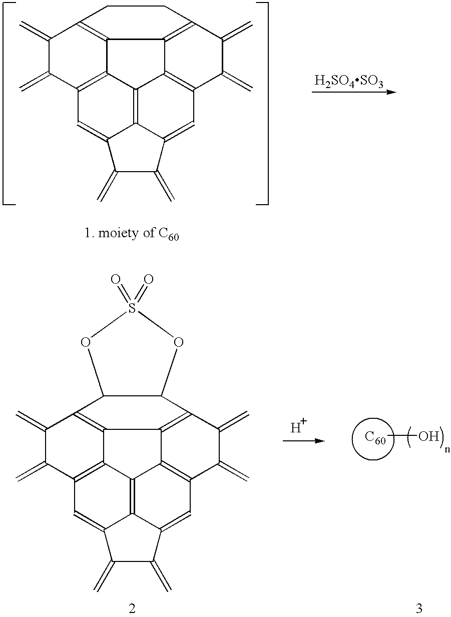

The polymers mentioned above are non-limiting examples of preferred embodiments, and any polymer or derivative that is capable of participating in electrostatic self-assembly may be used in the present invention.

The above-mentioned starting materials in the invention are used in an electrostatic self-assembly (“ESA”) process. Known ESA processes for constructing a thin film on a substrate may be used, such as ESA techniques previously used in certain non-biocompatible applications, for the synthesis of nonlinear optical thin films by polymer dyes, ceramic nanoparticle thin films, conductive thin films of metal nanoclusters, and light emitting diodes.

An ESA process may be performed at room temperature and can be used on substrates of arbitrary size and shape, which are advantageous features for easy manufacture. ESA processes generally proceed as follows: 1) providing a substrate; 2) optionally modifying the substrate to create a surface charge; 3) dipping the substrate into a charged inorganic cluster solution; 4) rinsing the substrate with solution; 5) dipping the substrate into an oppositely charged polymer solution; 6) rinsing the substrate with solution; 7) optionally repeating steps 3) to 6) to yield a multilayer coated substrate. The solutions in step 7) can be the same as, or different from the oppositely charged molecular solutions used in steps 3) to 6), or the mixture of two or more clusters or inorganic, organic or polymer molecules. The resulting multilayer coatings may consist of different blocks of inorganic clusters and polymer (or organic molecules).

By “clusters”, reference is made to substances that are not molecules, that are not chemically complete substances, and that may vary in size. Clusters preferably have sizes smaller than 30 nm.

Also, as an example of an ESA technique for the formation of multilayer thin films is one which allows detailed structural control at the molecular level with ease of manufacturing, see G. Decher, J. Schmitt, “Buildup of ultrathin multilayer film by a self-assembly process: III. Consecutively alternating adsorption of anionic and cationic polyelectrolytes on charged surfaces,” Thin Solid Films 210/211, 813-815, 1992.

Nanoclusters of fullerenes, so-called “Buckeyballs”, may be formed into multilayer thin films as described in J. Org. Chem. 1994, 59, 4960.

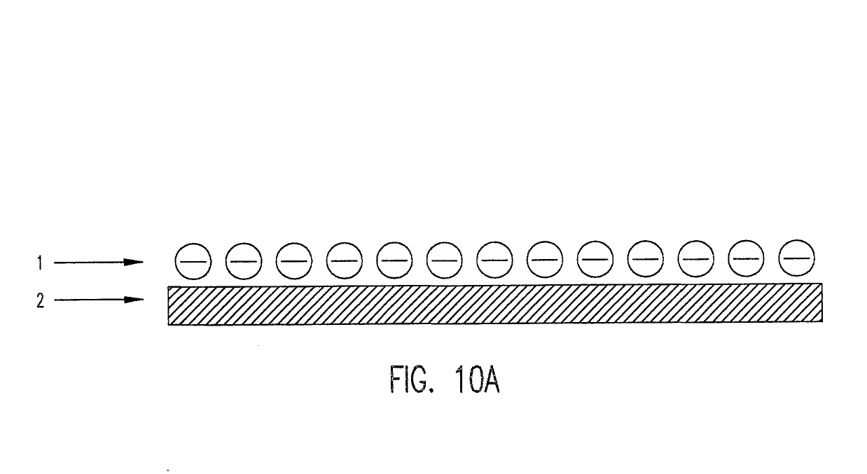

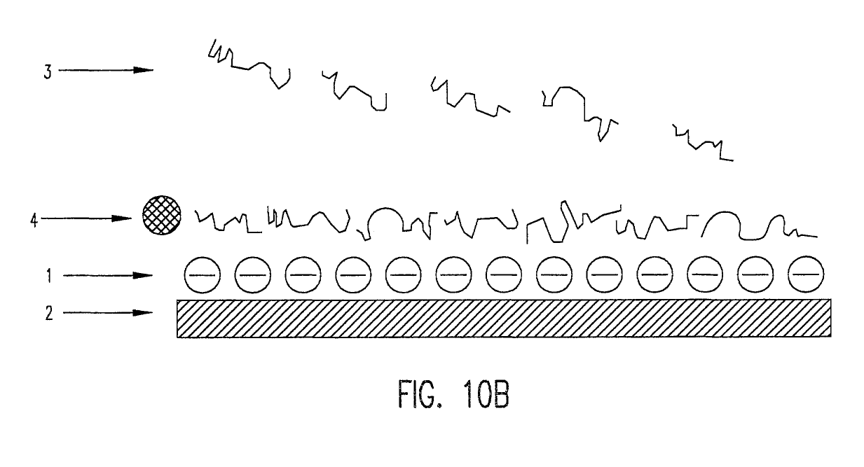

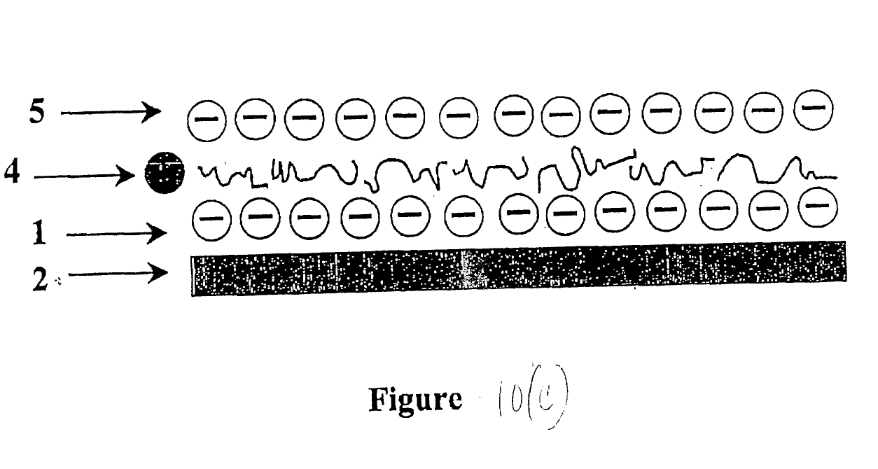

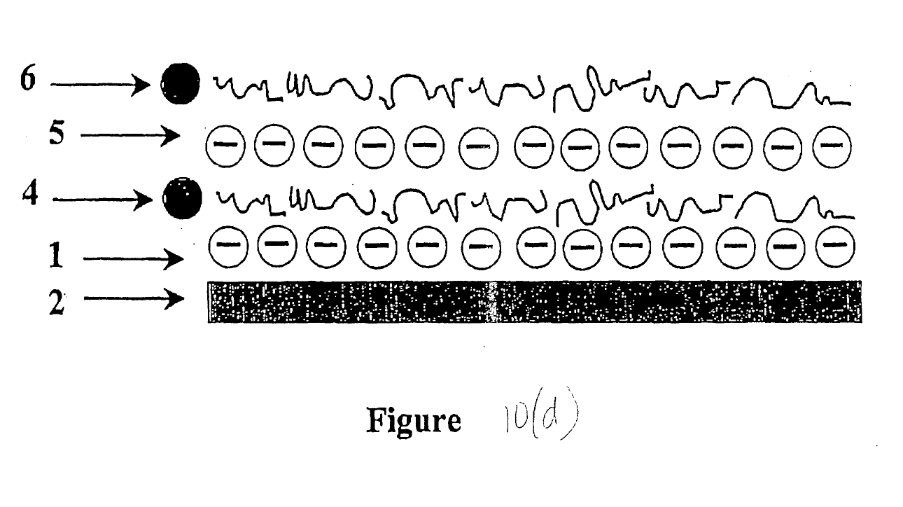

Referring now to the drawings, and more particularly to FIGS. 10(a) to (d), an example of an ESA thin-film fabrication process for use in the invention is as follows. A plastic substrate 1 is cleaned to remove surface impurities and to create a net charge 2 at the molecular surface of the substrate. The net charge region is shown as negative in FIG. 10(a) by way of example, but may be negative or positive. Although the substrate 1 is shown as flat in FIG. 10(a), it is not required that the substrate be flat or have any particular surface contour or shape.

Referring now to FIG. 10(b), there is shown the substrate 1, and net charge region 2, and cationic polymer molecules 3 that form a layer 4 on the substrate. Here, the polymer molecules are representative, and may be instead non-molecular clusters or other similarly sized materials with net positive outermost charge distributions.

FIG. 10(c) shows the substrate 1, the first layer of polymer molecules 4, and an additional negatively charged monolayer 5. Negatively charged clusters that are approximately spherical particles are shown, but in general different sizes, shapes and structures of negatively charged clusters may be used depending upon the method of their synthesis.

FIG. 10(d) shows the further addition of a second layer of polymer molecules 6, on top of the layer of clusters shown in FIG. 10(c). As in FIG. 10(b), these molecules have positive charges so they are cationic. Although the molecules 6 are shown as polymers, they may in general be clusters of positive charge or clusters of negative charge. Additionally, alternating layers of cluster and polymer molecules, or cluster and cluster, or cluster and other molecules may be added sequentially, where each layer has a charge opposite to that of the previously deposited layer. As long as this charge reversal is accomplished, the materials in the layers may be varied throughout the composite multilayer system.

It should be understood that this invention contemplates adding multiple layers of oppositely charged materials on top of each other in layer-by-layer fashion. The preferred aggregate thickness will vary depending on the materials used in the layers and on the application.

While FIG. 10(d) shows a negatively charged layer adhering to a positively charged substrate, it should be understood that the reverse arrangement also is within the scope of the invention. As discussed below, the actual production may be by sequentially dipping the substrate into baths containing the charge particles or polymers. The substrate on which the layers are applied can be made of naturally charged material, or can be treated to produce a charged surface (e.g., by chemical exposure, etching, plasma, etc.).

U.S. Pat. No. 6,114,099, which is herein incorporated by reference, describes the self-assembly of multilayered films, and these techniques can be used in this invention. U.S. Pat. No. 6,114,099 also describes patterned multi-layers. It will be appreciated that the film coating may be applied selectively and that the entire surface of the substrate is not required to be coated. For example, when the substrate to be coated is a urinary catheter, preferably only the catheter tip to be inserted into the body is coated.

While several specific materials are identified as being useful in forming a multi-layered coating on a substrate to render the device (e.g., implantable component, drug delivery device, catheter, etc.) Biocompatible with the biological material (e.g., cells, tissue, organ, bodily fluids, etc.) with which the device will come into contact, other materials might also be used to enhance biocompatibility within the scope of this invention. AU that is really required is that in the multilayered coating which coats all or a portion of the substrate, adjacent layers are held together by ionic attraction (referring to electrostatic attraction, ionic bonding or any other phenomenon which involves multiple positive and negative charges in adjacent layers holding the layers together), and at least one layer is relatively more biocompatible than the material or materials used in the substrate.

It will be appreciated that the ESA processes described herein by way of example are not limiting, and that modifications and variations may be made.

The present invention may use an ESA method that proceeds with alternate dipping of a charged substrate into aqueous solutions of oppositely-charged ions at room temperature. Such an ESA process allows ultra low-cost manufacturing, using simple dipping with alternating ionic molecules at room temperature, and fabrication of thin films on nearly any solid material substrate, including plastics, ceramics, metals or tissues, without degrading or destroying the substrates. It provides uniform thin films with any size and shape. Additionally, the thin films formed by ESA process on the substrate will provide a charged surface, and may improve adherence with osteoblasts, bone-forming cells and other cells.

In a preferred embodiment of a process according to the present invention, to obtain layer-by-layer construction of a thin film, a substrate is dipped into a solution containing the polymer or fullerene starting material. To obtain uniform thin films, concentration and pH value of solutions are carefully controlled during the dipping process.

The present inventors have found that for solutions containing C₆₀, preferably the concentration of the C₆₀ solution should be below 5×10⁻⁴ M, because aggregation will occur at a high concentration of C₆₀.

The substrate useable in the present invention is not particularly limited and may be any object or substance suitable for receiving a biocompatible coating, with the object or substance being in any shape and having any surface contour that will receive a thin-film coating. The substrate may be a titanium alloy, preferably Ti₆Al₄V. A substrate suitable for bone implant may be used. Bioactive glass may be used as the substrate. The substrate may consist essentially of a polymer, preferably, polyester. The invention in a preferred embodiment provides that the substrate is quartz. In other preferred embodiments, the substrate is glass, plastic, metal or ceramic. In another preferred embodiment, the substrate is suitable for tissue engineering.

In the invention, at least one ZrO₂, Al₂O₃ or TiO₂ metal oxide nanocluster may optionally participate in the electrostatic self-assembly. High purity of alumina (Al₂O₃) and zirconia (ZrO₂) are among the most widely used ceramics as biocompatible materials, and they are suitable for the fabrication of thin films by the ESA process due to their positive charge character in acidic conditions. Titania (TiO₂) is also considered highly biocompatible, and it is formed on the surface of titanium and its alloy. Also, titania nanoclusters may be synthesized by reaction of titanium tetrachloride (TiCl₄) with aqueous HCl solution to obtain a nanoparticle size of obtained TiO₂ of about 2 nm, measured by Transmission Electro Microscopy, and charged positively in pH of less than 3.

The present inventors have found that when a TiO₂ solution is used, the pH value preferably is adjusted to no more than 3; otherwise the precipitation of the metal oxide will affect the quality of the thin film.

Additionally, a wide variety of charged material may be used as alternating layers with nanoclusters, “Buckeyballs”, metal nanoclusters, ceramic nanoclusters, polyelectrolytes, and ionic polymers etc. within the practice of this invention so long as the optionally included material does not interfere with the biocompatibility of the thin film. It may be appreciated that a material may be non-biocompatible on its own, but could be included in a thin film according to the invention without destroying biocompatibility of the thin-film, and in such a case a film including such optionally-included material is within the present invention.

A film mono-layer constructed according to the present invention generally has a thickness of about 0.1 to 100 nanometers. A film constructed according to the present invention may have any desired thickness, such as 0.1 nanometers to 100 micrometers, and may be comprised of up to hundreds or thousands of mono-layers. Any surface treatment having at least one layer would fall within the scope of this invention. The film thickness preferably is of thickness greater than about 1 nm.

In a most preferred embodiment of a process according to the invention, thin film fabrication is at room temperature.

The invention in a preferred embodiment provides a biocompatible composition containing a plurality of layers electrostatically self-assembled from a starting material. The starting material may be poly(vinylpyrrolidone), poly{bis(carboxylatophenoxy)phosphazene}, poly(methacrylic acid), poly(l-lysine), poly(ethylene glycol), poly(D-glucosamine), poly(l-glutamic acid), poly(diallyldimethylamine), poly(ethylenimine), hydroxy fullerene or long-sidechain fullerene, or a combination thereof.

The biocompatible composition in addition to the starting material may contain any other material that does not interfere with its biocompatibility. Preferably, such biocompatible compositions and biocompatible materials according to the invention are formed as or into a thin film. Most preferably such a thin film is used to make a substrate biocompatible.

Advantageously, construction by electrostatic self-assembly provides a thin film that is uniform and homogeneous.

In another preferred embodiment the invention provides a drug delivery device, comprising a substrate made biocompatible by a process according to the invention and at least one drug. The drug may be incorporated as one or more layers within the multilayer structure, or could be associated with the surface layer of the multilayer structure.

Also, in a further preferred embodiment, the invention provides a medical device made by a process according to the present invention.

The medical device may be one onto which tissue for transplants may be engineered via the biocompatible coating surface of the device, onto which may be seeded cells that have been harvested from a specific organ.

In the medical device, the thin film may have an exposed surface (i.e., the surface not directly contacting the substrate) that has a charge to increase cell adhesion for cell growth.

In a medical device according to the present invention, the substrate is not particularly limited and may be tubing used in dialysis, tubing used in heart lung machines, other plastic tubing, other rubber tubing, bandaging material, composite material, metal material, insulator material, semi-conductor material, artificial hips, titanium substrates, pacemakers, plastic substrates, catheter material, stent material, and other materials used in medical devices.

It further will be appreciated that thin film coatings made according to ESA processes of the present invention may have a multi-functional nature, and the present invention uses such a multi-functional nature to advantage. For example, a coating according to the present invention may frustrate several different blood coagulation mechanisms, an advantageous feature with respect to stents.

In a preferred embodiment, the invention provides a device for contacting a biological material, comprising a substrate; and a multilayered coating positioned on at least a portion of a surface of said substrate wherein adjacent layers of said multilayered coating are held together by ionic attraction, and wherein at least one layer of said multilayered coating is made from a material that is relatively more biocompatible than a substrate material in said substrate, whereby said multilayer coating renders the device biocompatible with said biological material. The at least one layer may be, e.g., poly(vinylpyrrolidone), poly{bis(carboxylatophenoxy)phosphazene}, poly (methacrylic acid), poly(l-lysine), poly(ethylene glycol), poly(D-glucosamine), poly(l-glutamic acid), poly(diallyldimethylamine), poly(ethylenimine), hydroxy fullerene, long-sidechain fullerene. In such a device, the multilayered coating may include greater than 10 individual layers. The multilayered coating may include at least two layers made from different materials.

In another preferred embodiment, the invention also provides a method of rendering a device biocompatible with a biological material. Such a method may be performed by applying a multilayered coating on at least a portion of a surface of a substrate wherein adjacent layers of said multilayered coating are held together by ionic attraction, and wherein at least one layer of said multilayered coating is made from a material that is relatively more biocompatible than a substrate material in said substrate.

The present inventors made and tested ESA multilayer films according to the invention, including contact angle surface characterization, and in vitro protein adsorption studies with bovine albumin using Fourier Transform Infrared Reflection-Absorption Spectroscopy (FT-IRAS). The results indicate that the nanocomposite thin films fabricated with biomaterials by ESA processing may have broad potential applications for cell attachment and growth in tissue engineering.

Experimentation and testing were performed as follows.

All chemicals were reagent or HPLC grade. Alumina (Al₂O₃) and zirconia (ZrO₂) were purchased from Alfa Aesar, poly (vinylpyrrolidone) (PVP), poly(diallyldimethylamine) (PDDA), poly(ethylenimine) (PEI), fullerene (fullerite, a mixture C₆₀ and C₇₀), fuming sulfuric acid, and titanium tetrachloride were purchased from Aldrich, and Poly(methacrylic acid) (PMA) was purchased from Polysciences Inc. Titania (TiO₂) and polyhydroxylated fullerene were synthesized in our laboratory. Quartz was purchased from EL-CAT, Inc. Bovine serum albumin (BSA) was obtained from Alfa Aesar and used without any purification. The ultrapure water was obtained from a Barnstead Nanopure III system. FT-IR spectra were taken with a BIO-RAD FTS 6000 spectrometer equipped with a high sensitivity mercury-cadmium-telluride detector, and UV-Vis spectra were recorded on a Hitachi Model U-2001 spectrometer. An atomic force microscope (AFM) Digital Instruments Dimension™ 3100 was used to provide images of the fabricated thin films. Measurements of water contact angle of thin films were performed on a contact angle goniometer, Rame-Hart, Inc.

Synthesis of Titania

Titanium tetrachloride (99.9%, 44 ml) was added into aqueous HCl solution (2M, 156 ml) very slowly with vigorous stirring at 0° C. to obtain the solution of Titania. The aqueous solution was diluted to 0.16 M with Milli-Q water and pH was adjusted to 2.5 by addition of aqueous NaHCO₃.

Synthesis of Functionalized Fullerenes

The mixture of fullerene 1 (0.50 g, 0.682 mmole) in fuming sulfuric acid (30% SO₃, 10 ml) was heated to 60° C. with stirring under N₂ for 3 days. The resulting suspension was then added dropwise to diethyl ether, and the obtained precipitate was separated, washed three times with ether and twice with either/acetonitrile (2:1 V/V) before being dried under vacuum to yield cyclosulfatic fullerene 2, a brown-orange solid (0.63 g). Hydrolysis of 2 (0.62 g) in water (10 ml) was carried out at 80° C. for 10 hrs with stirring under N₂ to give polyhydroxylated fullerene 3 (0.502 g, 0.555 mmole, 81.3% overall yield from fullerene): FT-IR cm⁻¹ 3320 (br, s, OH), 1640 (s, C═C), 1044 (s, C—O—C).

ESA Fabrication of Biocompatible Thin-Films and Characterization

Multi-layer thin films of synthetic polydyes were self-assembled on quartz substrates with various biocompatible materials. The concentrations of aqueous solutions used for dipping processes were as followings: Al₂O₃ (20 mg/ml); ZrO₂ (20 mg/ml); TiO₂ (0.16 M); PMA (0.01 M); PVP (0.1 M); polyhydroxylated fullerene (2.5×10⁻⁴ M). Milli-Q water was used for all experiments and for all cleaning steps. Quartz substrates were treated with a mixture of hydrogen peroxide (H₂O₂) and concentrated sulfuric acid (3:7 V/V) for two hours and then washed with Milli-Q water before using in the ESA process. Each substrate was immersed for a specified time in the positively charged solution of material, rinsed with water, then immersed for a specified time in the negatively charged solution of material. The dipping process can be repeated as many time as desired. UV-Vis spectroscopy was used to identify the absorption and transmission characteristics of the thin-films as well as to quantify the growth of the multilayer structures. The conditions of the ESA processes are summarized in Table 1.

TABLE 1
ESA conditions for the fabrication of thin films.

| | pH values of the | Dipping | |
|---|---|---|---|
| Materials | solution | time (min) | pH value of rinse |
| PVP/PMA | 4.5 | 3.0 | 5.0 |
| PEI/PMA | 4.5 | 3.0 | 5.0 |
| Al₂O₃/PMA | 4.0 | 3.0 | 4.0 |
| ZrO₂/PMA | 3.5 | 3.0 | 3.0 |
| TiO₂/PMA | 9.0 | 3.0 | 3.0 |
| PDDA/C₆₀ | 4.0/5.5 | 2.0/5.0 | 9.0 |

Protein Adsorption on Thin Films

BSA, which is commonly used as a model blood plasma protein, was used. A 15 mg/ml solution of BSA in phosphate-buffered saline buffer (0.01 M, pH=7.4) was used within 3 hours. Thirty bilayer films were fabricated on the surface of a gold-coated glass (1 inch×1 inch) by the ESA process. The test thin film was immersed in BSA buffer solution for times as shown in Table 1, then washed with Milli-Q water and dried with nitrogen flow before IR spectra readings were taken.

Using a quartz and gold-plated glass substrate in each case, 30 bilayers were fabricated on each substrate according to the coating conditions in Table 1.

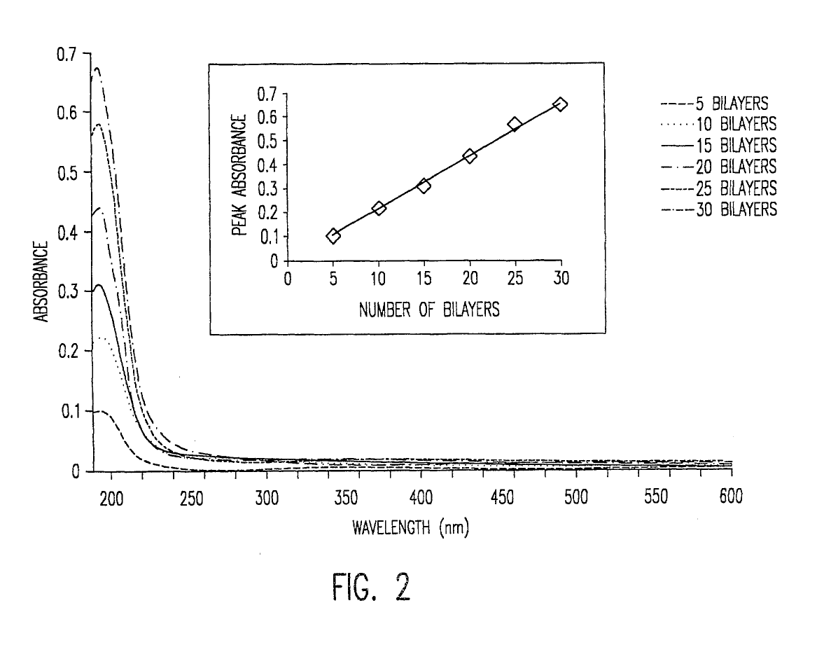

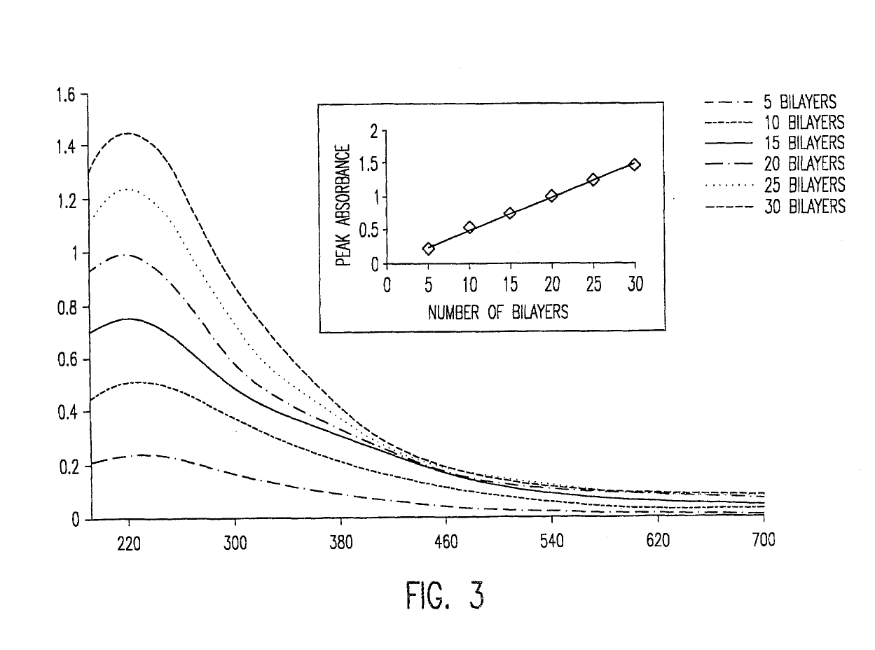

UV-Vis spectra were taken to monitor the ESA process of fabricated multi-layer thin films. Typical spectra are shown in FIG. 2 (PVP/PMA), and FIG. 3 (PDDA/C₆₀). FIG. 2 is a graph of UV-Vis spectra of a PVP/PMA ESA film, with results shown for 5, 10, 15, 20, 25 and 30 bilayers respectively. FIG. 3 is a graph of UV-Vis spectra of a VDDA/C₆₀ ESA film, with results shown for 5, 10, 15, 20, 25 and 30 bilayers, respectively.

With reference to FIGS. 2 and 3, in most cases, a linear increase with the addition of bilayers has indicated that each bilayer contributes an equal amount of material to the thin-film growth.

The surface imaging technique of AFM was used to characterize the produced thin-films, as to uniformity, grain distribution, and defect formation on the film surface.

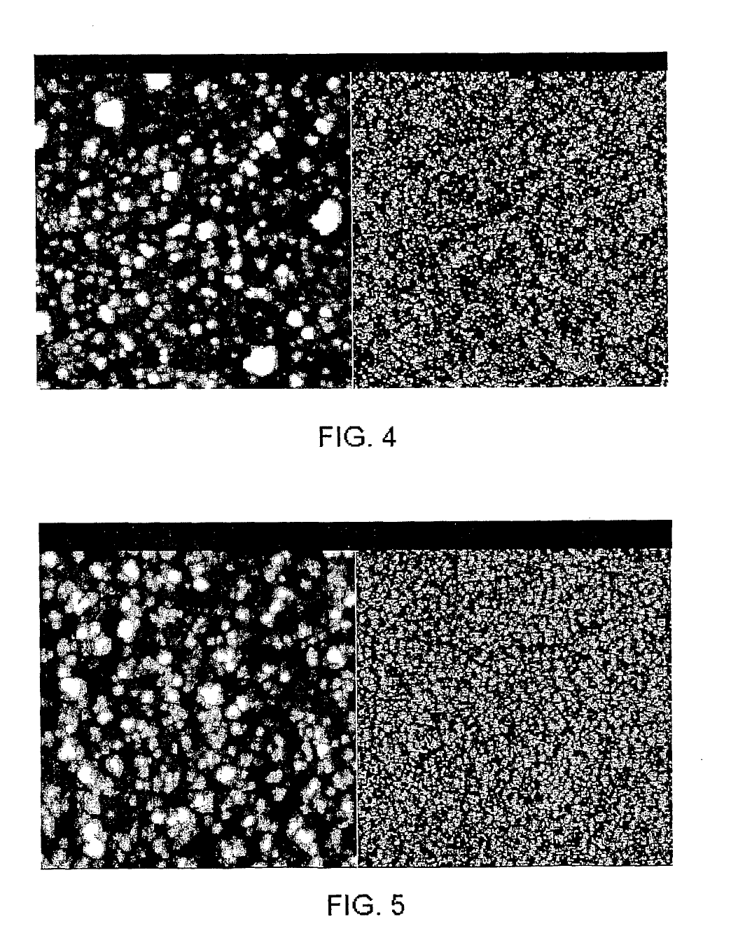

The AFM images of the ESA multilayer assemblies deposited on quartz substrates were obtained at ambient temperature. The pyramidal AFM tips and cantilevers were made from etched silicon probes. The images were collected in the tapping mode in air, resonating the tip just below the oscillation frequency of the cantilever (typically 250-325 kHz). The oscillation frequency for scanning was set to 0.1˜3 kHz below resonance. Typical images are shown in FIGS. 4 and 5.

FIG. 4 is an AFM image of a 30 bilayer PVP/PMA thin film. FIG. 5 is an AFM image of a 30 biloayer ZrO₂/PMA thin film. In each of FIGS. 4 and 5, the left view is a height image; the right view is a phase image. The images in FIGS. 4 and 5 were obtained with a scanning rate of 1 Hz and a data colletion resolution of 512×512 pixels. The images indicate that the films are uniform, showing no apparent surface damages or defects. Also in the image of FIG. 5, regular thin platelets of nanoclusters lying on the substrate plane can be observed; they are closely packed on the surface with an approximately uniform diameter of 20 nm. The present inventors believe that strong electrostatic interaction between the anionic and cationic monolayers results in the highly uniform nanostructure of the thin film.

Contact Angle Surface Characterization

Contact angles provide a measure of relative surface energies and thus may provide some indication of potential biocompatibility of a material. While contact angle information itself is not necessarily an indicator of biocompatibility, some possible correlations have been found between protein adsorption behavior and the water contact angle of material surfaces.

The measurement of water contact angle for 30-bilayer ESA thin films on quartz substrates was performed at three different locations on each specimen, with measurements shown in Table 2 below. The results may be interpreted with reference to the fact that surfaces with a higher concentration of oxygen atoms are known to exhibit higher wettability (smaller contact angle). From the experimental data shown in Table 2, the assembly pairs of Al₂O₃/PMA, ZrO₂/PMA, and TiO₂/PMA, in which high oxygen content is caused by the —COOH and the oxygen atoms in the metal oxide, have water contact angles less than 30°. On the contrary, PVP, PDDA, and PEI do not have those oxygen-based functional groups, and this fact may be the reason why the three polymer assembly pairs did not show good wettabilities. These observations from the contact angle measurements provide information about the surface characteristics, which could be pertinent to the interactions between blood (or tissue) and the biomaterial surfaces.

TABLE 2
Contact angles of ESA multilayer films.

| | WATER CONTACT ANGLE θ |
|---|---|
| MULTILAYER ASSEMBLIES | (°) |
| PVP/PMA | 45 |
| PDDA/C₆₀ | 52 |
| PEI/PMA | 55 |
| Al₂O₃/PMA | 18 |
| ZrO₂/PMA | 25 |
| TiO₂/PMA | 28 |

Measurements of Protein Adsorption by FT-IRAS

Foreign material inside the body comes in contact with a variety of functional units of living organisms, one class of which is that of proteins. Proteins are an important class of functional units in living organisms, e.g., as structural building blocks of tissue, as vehicles for transport of elements such as oxygen and CO₂, and for the catalytic-enzymatic processes that are central to life. The adsorption behavior of proteins on solid surfaces presently is much researched, because it plays a critical role in processes such as protein binding to cell surface receptor, biocompatibility of clinical implants, and solid-phase immunoassays. Aspects of protein surface adsorption include thermodynamic issues, which include hydrophobic, electrostatic and the structural effects, and kinetic issues. If the transport of the protein to the surface is diffusion controlled,

Λ α C (D t)^{1/2},

where Λ is the amount of protein present on the surface, C is the protein concentration in solution, D is the protein diffusion coefficient, and t is the time.

It is believed that the initial adsorption of protein from blood onto the surface of an implant or other foreign object in the body is one of the first events following implantation. The biocompatibility of materials is determined to a high degree by the characteristics of the formation of the irreversibly adsorbed protein layer following the initial stage of foreign-body contact, namely the rate of the formation, thickness, and the conformation of the adsorbed proteins. In vitro evaluations of protein adsorption on biomaterial surfaces have been prescribed by the National Institutes for Health Guidelines for Blood-Material Interactions as a partial indicator of biocompatibility. Guidelines for Blood-Material Interactions, NIH Publication No. 85-2185 (1985). The dominant mechanism in cellular attachment to a biomaterial surface is electrostatic in nature, with the electrostatic characteristics of the surface encouraging the adsorption of special proteins to facilitate initial attachment to the biomaterial surface. Protein adsorption to the solid surface plays a critical role in processes such as protein binding to cell surface receptors, the biocompatibility of clinical implants, and solid-phase immunoassays. Protein adsorption on biomaterial surfaces has become one of most widely used measuring sticks in biocompatibility analysis.

Accordingly, the present inventors proceeded to measure protein adsorption characteristics of thin films assembled by ESA according to the present invention, particularly using infrared reflection-absorption spectroscopy (IRAS). IRAS is an external reflection technique especially useful for the characterization of organic thin films on highly reflecting (non-transparent) solids such as metals and doped semiconductors. This technique may be used to characterize highly organized and anisotropic monolayers and ultra thin films. The sensitivity of WAS is very high, typically 0.1-1 monolayers, depending on the molecular system, making it possible to study adsorbate-monolayer interaction phenomena.

Using IRAS, the present inventors investigated the protein adsorption behavior of six different ESA multilayer assemblies: PVP/PMA, PDDA/C₆₀, PEI/PMA, Al₂O₃/PMA, ZrO_{2/}PMA, and TiO₂/PMA. The IRAS spectra were recorded before and after albumin adsorption for each sample. The spectra before the adsorption were subtracted from the spectra obtained afterwards, and the subtracted spectra give the net result of protein adsorption on the film surface. The increase in the area under the IRAS absorbance peak can be used to determine the adsorbed amounts, and any changes in the shape of the peak can be correlated to structural changes in the molecules of the adsorbed layer. Three of the assembly pairs, PVP/PMA, PDDA/C₆₀, and PEI/PMA, showed the albumin amide absorbance peak in the subtracted infrared spectra at frequencies around 1580 cm⁻¹˜1630 cm⁻¹. The amide band position showed obvious differences for spectra obtained from different surfaces. These shifts are probably caused by variations in conformational changes that occur after the protein is adsorbed onto the different solid surfaces. Table 3 below lists the amide frequencies for the adsorbed albumin (BSA) on these three ESA-formed thin film surfaces.

TABLE 3

| SURFACES | AMIDE FREQUENCY (cm⁻¹) |
|---|---|
| PVP/PMA | 1628 |
| PDDA/C₆₀ | 1588 |
| PEI/PMA | 1593 |

However, again referring to Table 3, an amide peak was not observed for the other three assemblies (Al₂O₃/PMA, ZrO₂/PMA, and TiO₂/PMA), which indicates that no detectable albumin adsorption occurred on those surfaces. Studies have shown that protein adsorption and cell adhesion is enhanced on a surface with a moderate wettability, but is prohibited on some hydrophilic surfaces. These later three film surfaces are all hydrophilic surfaces with contact angles less than 30°, while the wettabilities of the PVP/PMA, PDDA/C₆₀, and PEI/PMA surfaces are in the moderate range (contact angles are about 40°-50°). The results from protein adsorption are thus probably due to the effect of surface wettability. Another possible reason is that the presence of amino groups on the PVP/PMA, PDDA/C₆₀ and PEI/PMA film surfaces may assist albumin adsorption. These results suggest a structural aspect of protein adsorption on surfaces.

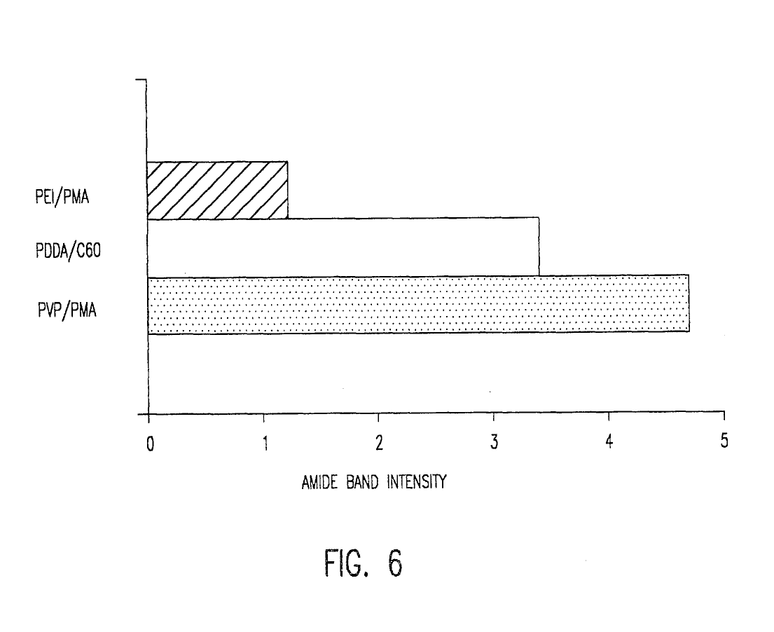

The thin films according to the invention are further characterized by FIG. 6, which shows amide band intesnity from the IRAS spectra after 1-hour albumin adsorption on different ESA thin films according to the invention. Among these three films, which did adsorb significant amounts of albumin, the PVP/PMA ESA surface adsorbed the highest amount, and PEI/PMA adsorbed the least.

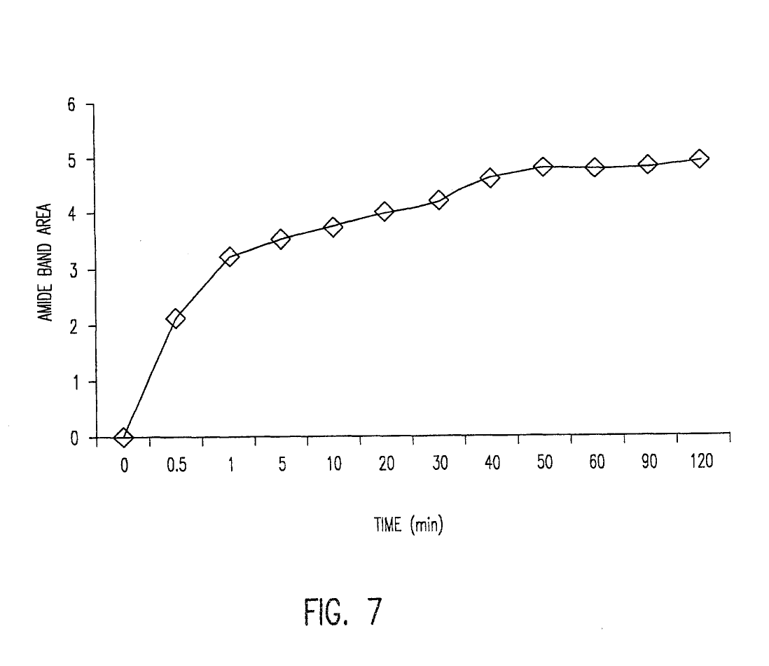

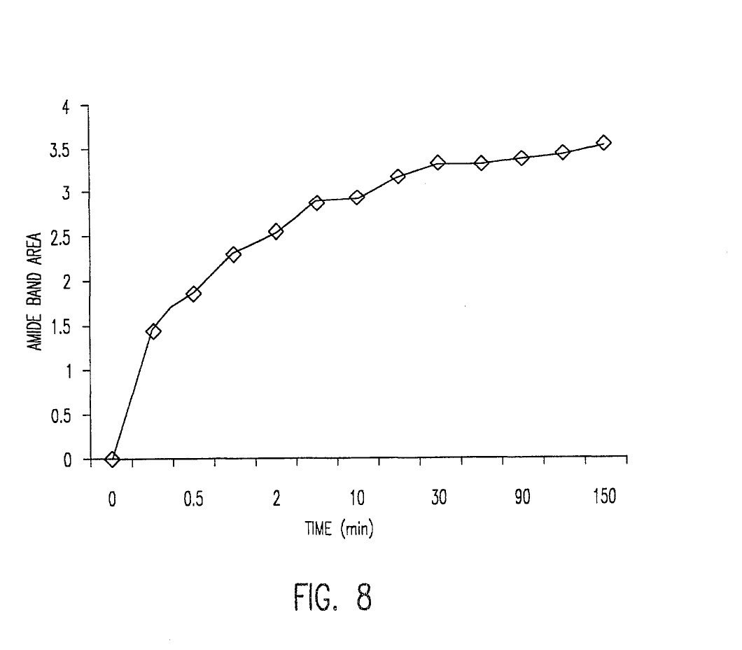

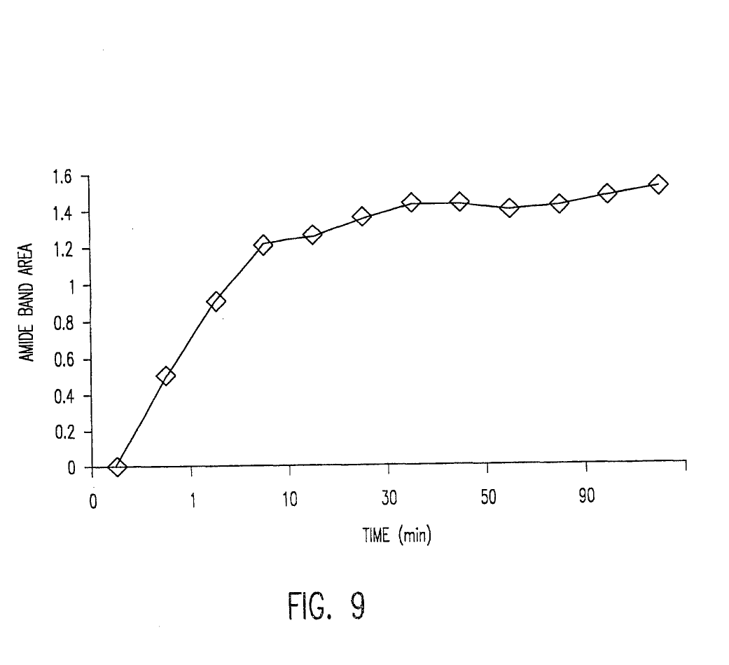

Kinetic studies were performed for different adsorption times for the coated substrates in the albumin solution on the ESA thin films. Individual IR band intensities were plotted versus time as shown in FIGS. 7, 8 and 9 respectively, each of which is a kinetic plot of albumin adsorption onto surfaces according to the invention. FIG. 7 is for a PVP/PMA film surface; FIG. 8 is for a PDDA/C₆₀ film surface; FIG. 9 is for a PEI/PMA surface. The amide band intensities were calculated from the integrated areas under the amide band absorbance peaks in each spectrum.

As seen from FIGS. 7, 8 and 9, on PVP/PMA and PDDA/C₆₀ film surfaces, initial albumin adsorption is extremely rapid for approximately 30 seconds after which it begins to slow down. The PEI/PMA film surface adsorbed albumin at a slower speed than the other two surfaces, and the amount of adsorbed albumin was also much less. After approximately 5-10 minutes, the adsorption nearly reached plateau in each tested case. Such behavior suggests rate limited diffusion behavior similar to that of oxidation processes.

From the data discussed above and with reference to the Figures, it will be appreciated that an ESA technique can be applied to provide homogeneous thin films on substrates using biocompatible materials, including ceramics, polymers and polydhydroxylated C₆₀. Contact angle surface characterization and in vitro protein adsorption measurements performed on the synthesized ESA multilayer thin films confirmed the biocompatibility feature. FT-IRAS spectra were used to obtain the relative amounts of adsorbed protein, and to analyze the kinetic adsorption behavior on certain film surfaces, also confirming the biocompatibility of thin films according to the invention. The ESA film surfaces which have high wettability (Al₂O₃/PMA ZrO₂,/PMA and TiO₂/PMA) prohibited protein adsorption onto the surface of the coatings, while surfaces possessing a moderate wettability (PVP/PMA, PDDA/C₆₀ and PEI/PMA) rapidly adsorbed a certain amount of protein in the first several minutes after contact.

Although preferred embodiments of the invention use a substrate, it will be appreciated that biocompatible materials may be constructed using ESA techniques to coat a substrate which then may be removed after a film or coating of desired thickness has been grown.

It will be appreciated that implantation in the body is not the only use for biocompatible materials, and they also may be used, outside the body, such as in contact with biological materials.

While the invention has been described in terms of its preferred embodiments, those skilled in the art will recognize that the invention can be practiced with modification within the spirit and scope of the appended claims.

We claim:

1. A process of making a substrate biocompatible comprising the steps of: contacting at least a portion of a charged substrate with an oppositely charged starting material and by electrostatic self-assembly constructing a multi-layered film of alternating charged molecular layers on the substrate,
wherein the substrate comprises a material selected from the group consisting of: ceramics, glasses, metals, metal alloys, plastics, and polymers; and
wherein the starting material is selected from the group consisting of:
poly(vinylpyrrolidone),
poly{bis(carboxylatophenoxy)phosphazene},
poly(methacrylic acid),
poly(l-lysine),
poly(ethyleneglycol),
poly(D-glucosamine),
poly(l-glutamic acid),
poly(diallyldimethylamine),
poly(ethylenimine),
hydroxy fullerene, and
long-sidechain fullerene.

2. A process according to claim 1, wherein also participating in the electrostatic self-assembly is a metal oxide selected from the group consisting of ZrO₂, Al₂O₃ and TiO₂.

3. A process according to claim 1, wherein individual monolayer thickness is about 0.1 nm to 100 nm.

4. A process according to claim 1, wherein the contacting is by dipping the substrate into a solution.

5. A process according to claim 1, wherein the substrate comprises quartz.

6. A process according to claim 1, wherein the substrate is selected from the group consisting of glasses, plastic, metals and ceramic.

7. A process according to claim 1, wherein said constructing step is performed at room temperature.

8. A process according to claim 1, wherein the substrate is suitable for tissue engineering.

9. A process according to claim 1, wherein the substrate is a titanium alloy.

10. A process according to claim 9, wherein the titanium alloy is Ti₆A₁₄V.

11. A process according to claim 1, wherein the substrate is suitable for bone implant.

12. A process according to claim 11, wherein the substrate is bioactive glass.

13. A process according to claim 1, wherein the substrate consists essentially of a polymer.

14. A process according to claim 13, wherein the polymer is polyester.

15. A drug delivery device, comprising:
a substrate, wherein the substrate comprises a material selected from the group consisting of: ceramics, glasses, metals, metal alloys, plastics, and polymers;
the substrate made biocompatible by a process according to claim 1;
and at least one drug.

16. A medical device comprising:
at least one surface, wherein the surface comprises a material selected from the group consisting of: ceramics, glasses, metals, metal alloys, plastics, and polymers; and further wherein the surface is made biocompatible by the process of claim 1.

17. A medical device according to claim 16, further comprising cells seeded onto the surface.

18. A biocompatible composition consisting essentially of:
a substrate comprising a material selected from the group consisting of ceramics, glasses, metals, metal alloys, plastics, and polymers; and
deposited on the substrate a plurality of layers electrostatically self-assembled from a starting material selected from the group consisting of:
poly(vinylpyrrolidone),
poly{bis(carboxylatophenoxy)phosphazene},
poly(methacrylic acid),
poly(l-lysine),
poly(ethylene glycol),
poly(D-glucosamine),
poly(l-glutamic acid),
poly(diallyldimethylamine),
poly(ethylenimine),
hydroxyfullerene, and
long-sidechain fullerene.

19. A biocompatible composition comprising:
a substrate, the substrate comprising a material selected from the group consisting of ceramics, glasses, metals, metal alloys, plastics, and polymers; and
deposited on the substrate a plurality of layers electrostatically self-assembled from a starting material selected from the group consisting of:
poly(vinylpyrrolidone),
poly{bis(carboxylatophenoxy)phosphazene},
poly(methacrylic acid),
poly(l-lysine),
poly(ethylene glycol),
poly(D-glucosamine),
poly(l-glutamic acid),
poly(diallyldimethylamine),
poly(ethylenimine),
hydroxy fullerene, and
long-side chain fullerene.

20. A biocompatible composition of claim 19 wherein the plurality of layers electrostatically self-assembled is at least 100 layers.

21. A biocompatible composition according to claim 20, wherein the plurality of layers is of uniform thickness.

22. A biocompatible composition according to claim 21, wherein each individual layer in the plurality of layers has a thickness greater than about 1 nm.

23. A biocompatible medical device or drug delivery device comprising: a substrate comprising a material selected from the group consisting of ceramics, glasses, metals, metal alloys, plastics, and polymers; and, provided on the substrate, a thin film electrostatically self-assembled starting with a solution of at least one stating material selected from the group consisting of:
poly(vinylpyrrolidone),
poly{bis(carboxylatophenoxy)phosphazene},
poly(methacrylic acid),
poly(l-lysine),
poly(ethylene glycol),
poly(D-glucosamine),
poly(l-glutamic acid),
poly(diallyldimethylamine),
poly(ethylenimine),
hydroxy fullerene, and
long-side chain fullerene.

24. The biocompatible medical device or drug delivery device of claim 23, wherein at least one appropriately charged metal oxide nanocluster is included.

25. The biocompatible medical device or drug delivery device of claim 24, wherein ZrO₂ is included.

26. The biocompatible medical device or drug delivery device of claim 24, wherein Al₂O₃ is included.

27. The biocompatible medical device or drug delivery device of claim 24, wherein TiO₂ is included.

28. The biocompatible medical device or drug delivery device of claim 23, wherein the thin film is prepared from a water-soluble polymer.

29. The biocompatible medical device or drug delivery device of claim 28, wherein the thin film is prepared from poly(vinylpyrrolidone).

30. The biocompatible medical device or drug delivery device of claim 28, wherein the thin film is prepared from poly{bis(carboxylatophenoxy)phosphazene}.

31. The biocompatible medical device or drug delivery device of claim 28, wherein the thin film is prepared from poly(methacrylic acid).

32. The biocompatible medical device or drug delivery device of claim 28, wherein the thin film is prepared from poly(l-lysine).

33. The biocompatible medical device or drug delivery device of claim 28, wherein the thin film is prepared from poly(ethylene glycol).

34. The biocompatible medical device or drug delivery device of claim 28, wherein the thin film is prepared from poly(D-glucosamine).

35. The biocompatible medical device or drug delivery device of claim 28, wherein the thin film is prepared from poly(l-glutamic acid).

36. The biocompatible medical device or drug delivery device of claim 28, wherein the thin film is prepared from poly(diallyldimethylamine).

37. The biocompatible medical device or drug delivery device of claim 28, wherein the thin film is prepared from poly(ethylenimine).

38. The biocompatible medical device or drug delivery device of claim 28, wherein the thin film is prepared from hydroxy fullerene.

39. The biocompatible medical device or drug delivery device of claim 28, wherein the thin film is prepared from long-side chain fullerene.

40. The biocompatible medical device or drug delivery device of claim 23, wherein the thin film has a surface not contacting the substrate that has a charge to increase cell adhesion for cell growth.

41. The biocompatible medical device or drug delivery device of claim 23, wherein the substrate is tubing used in dialysis.

42. The biocompatible medical device or drug delivery device of claim 23, wherein the substrate is tubing used in heart lung machines.

43. The biocompatible medical device or drug delivery device of claim 23, wherein the substrate is plastic tubing.

44. The biocompatible medical device or drug delivery device of claim 23, wherein the substrate is rubber tubing.

45. The biocompatible medical device or drug delivery device of claim 23, wherein the substrate is bandaging material.

46. The biocompatible medical device or drug delivery device of claim 23, wherein the substrate is composite material.

47. The biocompatible medical device or drug delivery device of claim 23, wherein the substrate is metal material.

48. The biocompatible medical device or drug delivery device of claim 23, wherein the substrate is insulator material.

49. The biocompatible medical device or drug delivery device of claim 23, wherein the substrate is semi-conductor material.

50. The biocompatible medical device or drug delivery device of claim 23, wherein the substrate is an artificial hip.

51. The biocompatible medical device or drug delivery device of claim 23, wherein the artificial hip comprises titanium.

52. The biocompatible medical device or drug delivery device of claim 23, wherein the substrate is a pacemaker.

53. The biocompatible medical device or drug delivery device of claim 23, wherein the pacemaker includes plastic.

54. The biocompatible medical device or drug delivery device of claim 23, wherein the substrate is a catheter.

55. The biocompatible medical device or drug delivery device of claim 23, wherein the substrate is a stent.

56. A process of making a substrate biocompatible comprising the steps of:
contacting at least a portion of a charged substrate, the substrate comprising a material selected from the group consisting of ceramics, glasses, metals, metal alloys, plastics, and polymers, with an oppositely charged starting material; and then
by electrostatic self-assembly, constructing a multi-layered film of alternating charged molecular layers on the substrate, wherein the starting material is a polymer.

57. The process according to claim 56, wherein also participating in the electrostatic self-assembly is a metal oxide selected from the group consisting of ZrO₂, Al₂O₃ and TiO₂.

58. The process according to claim 56, wherein the multi-layered film has a thickness of from about 0.1 nm to 100 nm.

59. The process according to claim 56, wherein the contacting is by dipping the substrate into a solution.

60. The process according to claim 56, wherein the substrate is quartz.

61. The process according to claim 56, wherein the substrate is selected from the group consisting of glasses, plastic, metals and ceramic.

62. The process according to claim 56, wherein the constructing step is performed at room temperature.

63. The process according to claim 56, wherein the substrate is suitable for tissue engneering.

64. The process according to claim 56, wherein the substrate is a titanium alloy.

65. The process according to claim 64, wherein the titanium alloy is Ti₆A₁₄V.

66. The process according to claim 56, wherein the substrate is suitable for bone implant.

67. The process according to claim 66, wherein the substrate is bioactive glass.

68. The process according to claim 56, wherein the substrate consists essentially of a polymer.

69. The process according to claim 68, wherein the polymer is polyester.

70. A drug delivery device, comprising:
a substrate comprising a material selected from the group consisting of ceramics, glasses, metals, metal alloys, plastics, the substrate made biocompatible by a process according to claim 56; and
at least one drug.

71. A medical device comprising:
at least one surface, wherein the surface comprises a material selected from the group consisting of ceramics, glasses, metals, metal alloys, plastics, and polymers; and further
wherein the surface is made biocompatible by the process of claim 56.

72. A medical device according to claim 71, further comprising cells seeded onto the biocompatible surface.

73. A biocompatible material consisting essentially of a plurality of layers electrostatically self-assembled from a starting material that is a polymer, the layers deposited on a substrate comprising a material selected from the group consisting of ceramics, glasses, metals, metal alloys, plastics, and polymers; and further.

74. A biocompatible material comprising a plurality of layers electrostatically self-assembled from a sting material that is a polymer, the layers deposited on a substrate comprising a material selected from the group consisting of ceramics, glasses, metals, metal alloys, plastics, and polymers; and further.

75. The biocompatible material according to claim 74, wherein the plurality of layers electrostatically self-assembled is at least 100 layers.

76. The biocompatible material according to claim 75, wherein the thin film is uniform and homogeneous.

77. The biocompatible material according to claim 76, wherein the thin film is of thickness greater than about 1 nm.

78. The biocompatible material of claim 74, wherein at least one metal oxide nanocluster is included.

79. The biocompatible material of claim 78, wherein ZrO₂ is included.

80. The biocompatible material of claim 78, wherein Al₂O₃ is included.

81. The biocompatible material of claim 78, wherein TiO₂ is included.

82. A device for contacting a biological material, the device comprising:
a substrate comprising a material selected from the group consisting of ceramics, glasses, metals, metal alloys, plastics, and polymers; and
a multilayered coating positioned on at least a portion the substrate, wherein adjacent layers of the multilayered coating are held together by ionic attraction, and wherein at least one layer of the multilayered coating is made from a material that is more biocompatible than the material in the substrate, whereby the multilayer coating renders the device biocompatible with the biological material.

83. The device of claim 82, wherein said at least one layer is selected from the group consisting of:
poly(vinylpyrrolidone),
poly{bis(carboxylatophenoxy)phosphazene},
poly(methacrylic acid) poly(l-lysine),
poly(ethylene glycol),
poly(D-glucosamine),
poly(l-glutamic acid),
poly(diallyldimethylamine),
poly(ethylenimine),
hydroxy fullerene, and
long-sidechain fullerene.

84. The device of claim 83, wherein the multilayered coating includes greater than 10 individual layers.

85. The device of claim 83, wherein the multilayered coating includes at least two layers made from different materials.
